(19) United States
(12) Patent Application Publication
(10) Pub. No.: US 2025/0191823 A1
(43) Pub. Date: Jun. 12, 2025

(54) Medical-Device Magnetizer Systems and Methods

(71) Applicant: Bard Access Systems, Inc., Salt Lake City, UT (US)

(72) Inventors: Steffan Sowards, Salt Lake City, UT (US); Anthony K. Misener, Bountiful, UT (US); William Robert McLaughlin, Bountiful, UT (US)

(21) Appl. No.: 19/054,555

(22) Filed: Feb. 14, 2025

Related U.S. Application Data
(63) Continuation of application No. 17/872,814, filed on Jul. 25, 2022, now Pat. No. 12,230,427.
(60) Provisional application No. 63/225,793, filed on Jul. 26, 2021.

(51) Int. Cl. H01F 13/00; A61B 5/06; A61B 34/20

(52) U.S. Cl. CPC H01F 13/003; A61B 34/20; A61B 5/062

(57) ABSTRACT

A method of imparting a magnetic signature includes inserting a first medical device into a single-dipole section of a magnetizer and positioning a second medical device into a multipole section of the magnetizer. Inserting the first medical device into the single-dipole section can impart a single-dipole magnetic signature with a first magnetic field to the first medical device. Positioning the second medical device into the multipole section can impart a multipole magnetic signature with a second magnetic field to the second medical device.

### PRIORITY

This application is a continuation of U.S. patent application Ser. No. 17/872,814,filed Jul. 25, 2022, now U.S. Pat. No. 12,230,427, which claims the benefit of priority to U.S. Provisional Patent Application No. 63/225,793, filed Jul. 26, 2021, each of which is incorporated by reference in its entirety into this application.

### BACKGROUND

A ferrous medical device can be magnetized for the purpose of tracking the ferrous medical device in 3-dimensional (“3D”) space. The ferrous medical device can be brought into a magnetic field of a magnetizer, thereby imparting to the medical device a unique magnetic signature able to be tracked in 3D space. However, if multiple unique magnetic signatures are needed, multiple different magnetizers are required. It would be beneficial to have one magnetizer configured to provide multiple unique magnetic signatures.

Disclosed herein are medical-device magnetizer systems and methods that address the foregoing.

### SUMMARY

Disclosed herein is a magnetizer system configured to impart one or more magnetic signatures to a medical device having ferrous elements for medical-device tracking. The magnetizer system includes, in some embodiments, a magnetizer. The magnetizer has an elongate body with a single-dipole section, a multipole section, and a plurality of magnets configured to generate two or more magnetic fields. The single-dipole section has a magnetizer body defining a cavity configured to have a first magnetic field therein. The multipole section is configured to have a second magnetic field therein.

In some embodiments, the magnetizer includes one or more sensors configured to detect the medical device through optical sensing, inductive sensing, or a radiofrequency identification (“RFID”) reader configured to detect an RFID tag coupled to the medical device.

In some embodiments, the one-or-more sensors are configured to be in communication with a computing device or another medical device.

In some embodiments, the one-or-more sensors are configured to communicate the imparting of the one-or-more magnetic signatures to the medical device, the computing device, or the other medical device.

In some embodiments, the other medical device is an ultrasound probe.

In some embodiments, the first magnetic field is configured to impart a single-dipole magnetic signature. The second magnetic field is configured to impart a multipole magnetic signature.

In some embodiments, the multipole section includes two shoulders extending perpendicularly from the elongate body. The two shoulders define a trough configured to receive the medical device therein.

In some embodiments, the plurality of magnets are configured to impart the single-dipole magnetic signature within the cavity and the multipole magnetic signature within the trough.

In some embodiments, one or more magnets of the plurality of magnets are configured to impart the single-dipole magnetic signature within the cavity. One or more other magnets of the plurality of magnets are configured to impart the multipole magnetic signature within the trough.

In some embodiments, the one-or-more magnets of the plurality of magnets are located within the cavity.

In some embodiments, the one-or-more other magnets of the plurality of magnets are arranged in an array of magnets within the trough.

In some embodiments, one or both of the shoulders include a post extending therefrom. Each post is configured to guide a tray having the medical device in a horizontal orientation thereon into the trough.

In some embodiments, the tray having the medical device in the horizontal orientation thereon is configured to be lowered vertically into the trough and subsequently removed to impart the multipole magnetic signature to the medical device.

In some embodiments, the magnetizer is configured for the medical device to be slidably inserted into the cavity in a horizontal orientation and subsequently withdrawn from the cavity to impart the single-dipole magnetic signature to the medical device.

In some embodiments, the magnetizer is configured for the medical device to be slidably inserted into the cavity in a vertical orientation and subsequently withdrawn from the cavity to impart the single-dipole magnetic signature to the medical device.

In some embodiments, the single-dipole section and the multipole section are on opposite ends of the magnetizer.

In some embodiments, the single-dipole section and the multipole section are alongside each other in the magnetizer.

In some embodiments, the plurality of magnets include electromagnets.

In some embodiments, the magnetizer includes a mechanical switch configured to detect the medical device.

In some embodiments, the mechanical switch is configured to indicate whether the medical device includes any magnetic signature thereon.

Also disclosed herein is a method of imparting a magnetic signature to a plurality of medical devices having ferrous elements using a magnetizer system. The method includes, in some embodiments, slidably inserting a first medical device into a single-dipole section of a magnetizer body of a magnetizer to impart a single-dipole magnetic signature to the ferrous elements of the first medical device with a first magnetic field; placing a second medical device in a horizontal orientation in a multipole section of the magnetizer to impart a multipole magnetic signature to the ferrous elements of the second medical device with a second magnetic field; and removing the first and second medical devices from the magnetizer.

In some embodiments, slidably inserting the first medical device into the single-dipole section of the magnetizer body includes slidably inserting the first medical device in the horizontal orientation or a vertical orientation.

In some embodiments, the magnetizer body defines a cavity having one or more magnets of a plurality of magnets therein. The one-or-more magnets are configured to generate the first magnetic field.

In some embodiments, the multipole section of the magnetizer includes one or more other magnets of the plurality of magnets. The one-or-more other magnets are configured to generate the second magnetic field.

In some embodiments, the multipole section of the magnetizer includes two shoulders extending perpendicularly from the magnetizer. The two shoulders have the one-or-more other magnets of the plurality of magnets therein. The two shoulders also define a trough including the second magnetic field therein.

In some embodiments, placing the second medical device in the horizontal orientation in the multipole section of the magnetizer includes lowering the second medical device on a tray into the trough.

In some embodiments, removing the second medical device from the magnetizer includes removing the second medical device from the magnetizer in the vertical orientation from the multipole section by removing the tray from the trough.

In some embodiments, removing the first medical device from the magnetizer includes slidably removing the first medical device in the horizontal orientation from a cavity defined by the magnetizer body.

In some embodiments, removing the medical device from the magnetizer includes slidably removing the first medical device from the magnetizer in a vertical orientation from a cavity defined by the magnetizer body.

In some embodiments, removing the second medical device from the magnetizer includes withdrawing the second medical device from the magnetizer in the horizontal orientation from the multipole section of the magnetizer.

These and other features of the concepts provided herein will become more apparent to those of skill in the art in view of the accompanying drawings and following description, which describe particular embodiments of such concepts in greater detail.

### BRIEF DESCRIPTION OF DRAWINGS

FIG. 1 illustrates a perspective view of a magnetizer, in accordance with some embodiments.

FIG. 2 illustrates a side view of the magnetizer, in accordance with some embodiments.

FIG. 3A illustrates a cross sectional view of the magnetizer, in accordance with some embodiments.

FIG. 3B illustrates a cross sectional view of imparting a multipole magnetic signature or a single-dipole magnetic signature to the medical device, in accordance with some embodiments.

FIG. 3C illustrates a cross sectional view of imparting a multipole magnetic signature or a single-dipole magnetic signature to the medical device, in accordance with some embodiments.

FIG. 4 illustrates a flow chart of a method of imparting a multipole magnetic signature or a single dipole signature to the medical device, in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

The term “computing device” should be construed as electronics with the data processing capability and/or a capability of connecting to any type of network, such as a public network (e.g., Internet), a private network (e.g., a wireless data telecommunication network, a local area network [“LAN”], etc.), or a combination of networks. Examples of a computing device can include, but are not limited or restricted to, the following: a server, an endpoint device (e.g., a laptop, a smartphone, a tablet, a “wearable” device such as a smart watch, augmented or virtual reality viewer, or the like, a desktop computer, a netbook, a medical device, or any general-purpose or special-purpose, user-controlled electronic device), a mainframe, internet server, a router; or the like.

Unless defined otherwise, all technical and scientific terms used herein have the same meaning as commonly understood by those of ordinary skill in the art.

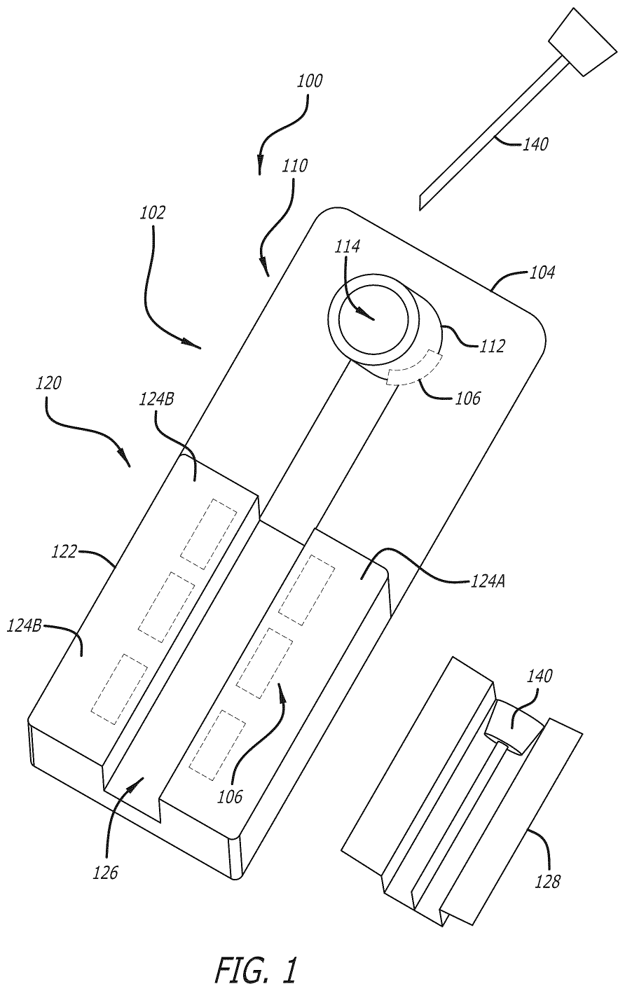

FIG. 1 illustrates a perspective view of a magnetizer system 100, in accordance with some embodiments. In some embodiments, the magnetizer system 100 includes a magnetizer 102 having an elongate magnetizer body 104 having a single-dipole section 110 and a multipole section 120. Each section is configured to include a magnetic field that imparts a unique magnetic signature on ferrous elements of a medical device 140 within the magnetic field for medical-device tracking. In some embodiments, the single-dipole section 110 and the multipole section 120 can each include a plurality of magnets 106 arranged in a configuration such as an array. In some embodiments, the plurality of magnets 106 can be shared between the single-dipole section 110 and the multipole section 120. The medical device 140 having the ferrous elements can be configured to be brought into the magnetic field to have the unique magnetic signature imparted thereto for medical-device tracking in 3D space.

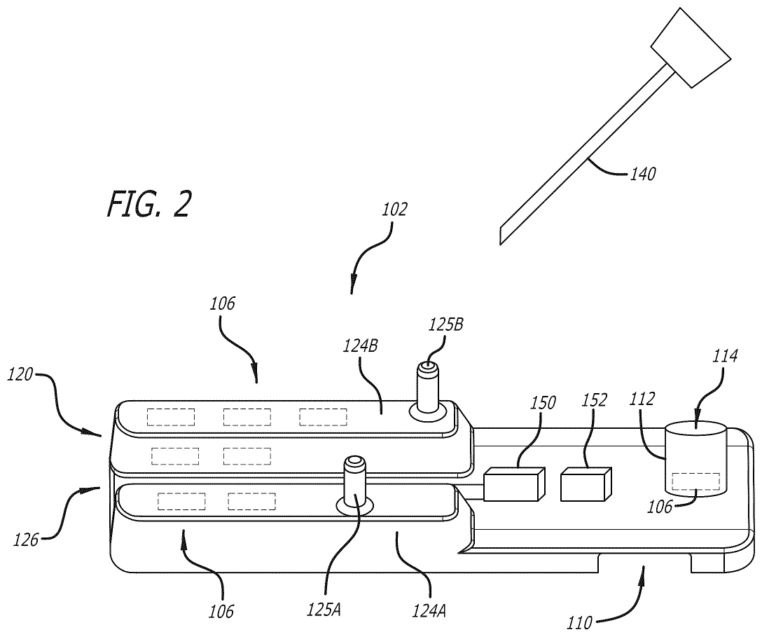

In some embodiments, the single-dipole section 110 can include a single dipole magnetizer 112 having the magnetizer body 104 configured as a magnetizer body that defines a cavity 114 such as tube extending from a surface of the magnetizer body 104 or into a shoulder of the shoulders 124A and 124B. In some embodiments, the single dipole magnetizer 112 can extend perpendicularly from the magnetizer body 104 as shown in FIG. 2. In some embodiments, the magnetizer body 104 can include one or more magnets from the plurality of magnets 106 therein configured to generate a magnetic field within the cavity 114. In some embodiments, the magnetic field within the cavity 114 can be configured to impart a single-dipole magnetic signature to ferrous elements therein. In some embodiments, the single dipole magnetizer 112 can be detachably coupled to the magnetizer body 104 through a press fit, a snap fit, an interference fit, or the like.

The multipole section 120 can include a multipole magnetizer 122, extending perpendicular from the magnetizer 102. In some embodiments, the multipole magnetizer 122 can include two elongated shoulders 124A and 124B extending perpendicularly from the magnetizer 102 (e.g., longitudinal walls of the shoulders 124A and 124B are perpendicular to a surface of the magnetizer 102), thereby defining a trough 126 along a central portion of a length of the magnetizer 102. In some embodiments, the trough 126 can be configured to receive a tray 128 therein. The tray 128 can include the medical device 140 having ferrous elements therein, allowing the user the ability to easily place the medical device 140 into and remove the medical device 140 from a multipole magnetic field. In some embodiments, one or more magnets of the plurality of magnets 106 can reside within the shoulders 124A and 124B, within the trough 126, or both, and the one-or-more magnets of the plurality of magnets 106 can generate the multipole magnetic field within the trough 126. Advantageously, the magnetizer 102 can be used to impart either a single-dipole magnetic signature or a multipole magnetic signature to the medical device 140 having ferrous elements instead of having multiple separate magnetizers to impart either the single-dipole magnetic signature or the multipole magnetic signature to allow for unique tracking depths and packaging configurations of the medical device 140.

FIG. 2 illustrates a side view of the magnetizer 102, in accordance with some embodiments. In some embodiments, the magnetizer 102 can be configured to detect the magnetic signature of the medical device 140. In some embodiments, the magnetizer 102 can include one or more sensors 150 configured to detect the presence of the medical device 140 or the magnetic signature of the medical device 140. In some embodiments, the one-or-more sensors 150 can be configured to be in communication with a computing device or another medical device. In some embodiments, the one-or-more sensors 150 can be in wireless communication with the computing device or the other medical device. Example wireless communication modalities can include Wi-Fi, Bluetooth, Near Field Communications (“NFC”), cellular Global System for Mobile Communication (“GSM”), electromagnetic (“EM”), radiofrequency (“RF”), combinations thereof, or the like. In some embodiments, the one-or-more sensors 150 can be configured to detect the status of imparting the one-or-more magnetic signatures to the medical device 140, wherein the status of imparting the one-or-more magnetic signatures to the medical device 140 can range from pending, initiating, currently imparting, and finished imparting. In such embodiments, the one-or-more sensors 150 can be further configured to communicate to the computing device or the other medical device the status of the imparting of the one-or-more magnetic signatures to the medical device 140. In some embodiments, the other medical device can include an ultrasound probe or the like. In some embodiments, the one-or-more sensors 150 can include one or more of: a RFID reader configured to detect a RFID tag coupled to the medical device 140, an optical sensor, or an inductive sensor. In some embodiments, the one-or-more sensors 150 can be in communication with a console, the console being in wireless communication with a computing device or another medical device. In some embodiments, the one-or-more sensors 150 can be coupled to the magnetizer 102, can be embedded within the magnetizer 102 or formed integrally into the magnetizer 102. In some embodiments, the magnetizer 102 can include a mechanical switch 152 configured to detect the presence of the medical device 140 or indicate that the medical device 140 includes a magnetic signature thereon.

In some embodiments, the plurality of magnets 106 can include passive magnets or active magnets (e.g., electromagnets). In some embodiments, the multipole section 120 can be configured to receive therein the medical device 140. In some embodiments, the multipole section 120 have include one or more posts 125A, 125B, . . . , 125n extending transversely or perpendicularly from the shoulders 124A and 124B. The one-or-more posts 125A, 125B, . . . , 125n can be configured to guide the tray 128 holding the medical device 140 therein into the trough 126, allowing the tray 128 to be precisely lowered and removed from the trough 126 vertically to ensure the unique multipole magnetic signature is imparted to the medical device 140.

In some embodiments, the single-dipole section 110 can be located opposite the multipole section 120 such as on opposite ends of the magnetizer 102. In some embodiments, the single dipole magnetizer 112 can extend transversely from the magnetizer 102. In some embodiments, the single dipole magnetizer 112 can be shaped in a tube or cylinder, a right prism such as a rectangular prism, or the like, thereby defining the cavity 114. The cavity 114 can include therein one or more magnets of the plurality of magnets 106, wherein the medical device 140, in a vertical orientation, can be slidably inserted into the cavity 114 and removed, to impart the single-dipole magnetic signature to the medical device 140. In some embodiments, the vertical orientation includes where the medical device 140 is perpendicular to the magnetizer 102 or when the single dipole magnetizer 112 extends perpendicularly from the magnetizer 102, the vertical orientation includes when the medical device 140 is parallel to the single dipole magnetizer 112.

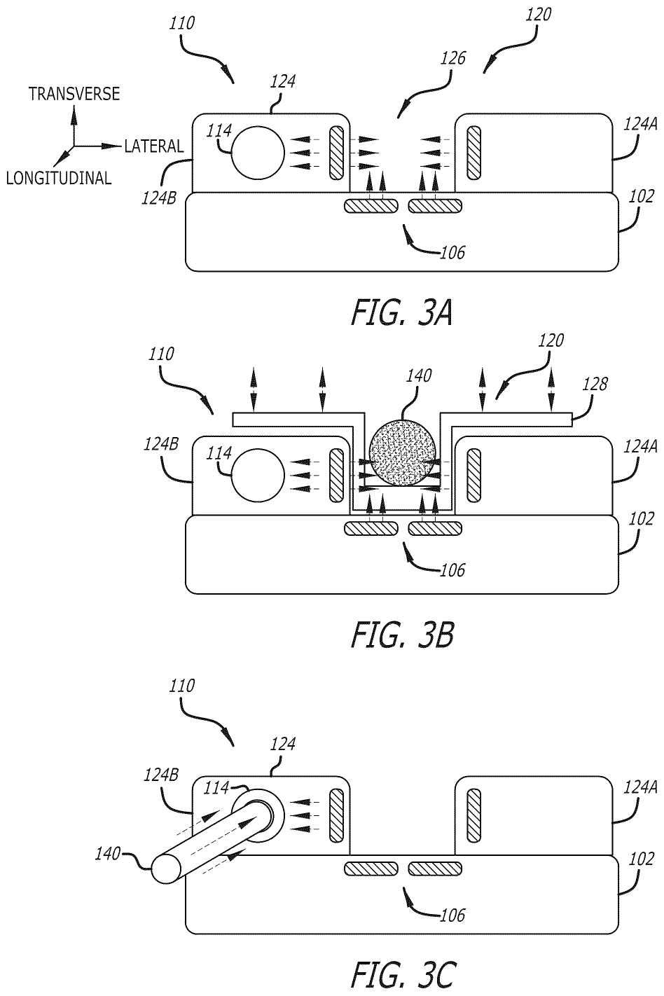

FIG. 3A illustrates a cross-sectional view of the magnetizer 102, in accordance with some embodiments. In an embodiment, the magnetizer 102 can include the trough 126. In this embodiment, the single-dipole section 110 and the multipole section 120 are alongside each other in the magnetizer 102, allowing the plurality of magnets 106 to generate both the single dipole magnetic field and the multipole magnetic field. The plurality of magnets 106 surround the trough 126 as illustrated in FIG. 3A and generate a multipole magnetic field within the trough 126. The plurality of magnets 106 can be configured to magnetize a portion of the medical device 140 by providing a multipole magnetization. In this embodiment, the magnetizer cavity 114 of the single dipole magnetizer 112 is defined as the tube or cylinder, the right prism such as the rectangular prism, or the like, in one shoulder of the shoulders 124A and 124B, wherein some of the plurality of magnets 106 generate a single dipole magnetic field within the cavity 114. In this embodiment, the cavity 114 can stretch along a portion up to an entirety of the shoulder 124A or 124B.

FIGS. 3B and 3C illustrate a cross-sectional view of magnetizing the medical device 140 with either a single dipole magnetization or a multipole magnetization, in accordance to some embodiments. In this embodiment, the medical device 140 can be placed in the tray 128 in the trough 126. The plurality of magnets 106 can be configured around the trough 126 so that the plurality of magnets 106 generates a multipole magnetic field therein. The medical device 140 in a horizontal orientation within the tray 128 can be lowered into the trough 126 wherein the multipole magnetic field magnetizes a portion of the medical device 140 with a multipole magnetic signature. In some embodiments, the horizontal orientation includes when the medical device 140 is parallel to the magnetizer 102. The tray 128 containing the medical device 140 can be removed from the trough 126 by lifting vertically while the medical device 140 is in the horizontal configuration. The medical device 140 having the magnetic signature thereon can then be tracked in 3D space by various means.

As illustrated in FIG. 3C, the magnetizer 102 includes the trough 126 and further includes the cavity 114. The cavity 114 can be located longitudinally in line with one or more magnets of the plurality of magnets 106. The plurality of magnets 106 can be configured to generate a single dipole magnetic field within the cavity 114. The plurality of magnets 106 can be configured to generate a single dipole magnetic field along at least a portion of the length of the cavity 114. The medical device 140 in a horizontal orientation can be inserted longitudinally into the cavity 114 and slidably removed from the cavity 114. As the medical device 140 is slidably removed from the cavity 114, the single dipole magnetic field imparts a single-dipole magnetic signature to a portion of the medical device 140.

Notably, the magnetic signature can be consistent an encoding of differentiating information or data for the medical device 140 such that the magnetic signature of a first instance of the medical device 140 is different from that of a second instance of the medical device 140. In some embodiments, the differentiating information or data can include model information for the medical device 140 such as a model name or number of the medical device 140. In some embodiments, the differentiating information or data can include dimensional information for the medical device 140 such as a length or diameter of the medical device 140. In some embodiments, the differentiating information or data can include manufacturing information for the medical device 140 such as a manufacturing date or lot number of the medical device 140. In some embodiments, the differentiating information or data can include unique information pertaining to the medical device 140 such as a serial number of the medical device 140. As such, in some embodiments, the magnetic signature for each instance of the medical device 140 can be unique with respect to that for every other instance of the medical device 140.

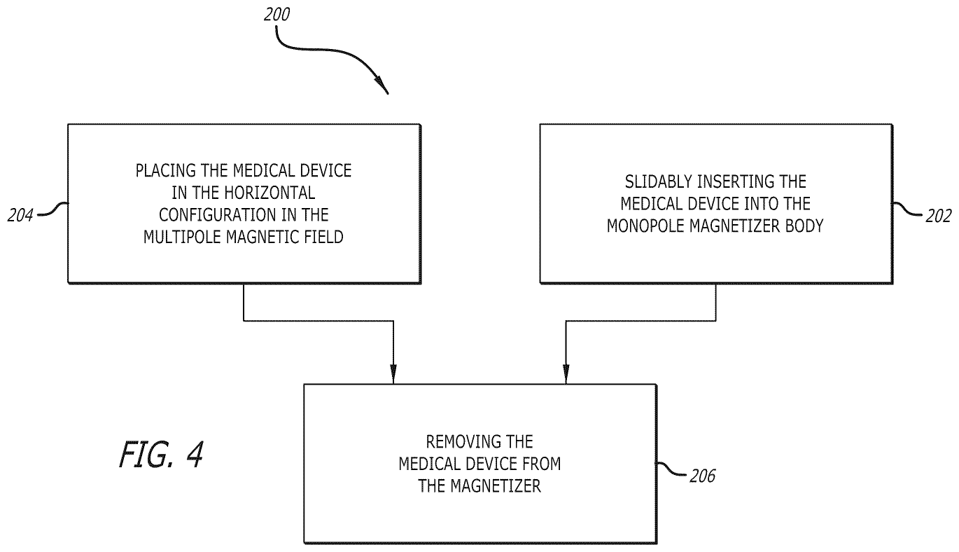

FIG. 4 illustrates a flow chart of a method 200 of imparting a magnetic signature to the medical device 140 or a plurality of such medical devices having ferrous elements, in accordance with some embodiments. In some embodiments, the method 200 further includes slidably inserting the medical device 140 or a first medical device of the plurality of medical devices in the single dipole magnetizer 112 to impart a single-dipole magnetic signature (block 202). In some embodiments, the single dipole magnetizer 112 includes the cavity 114 and one or more of the plurality of magnets 106 therein. In some embodiments, slidably inserting the medical device 140 or the first medical device of the plurality of medical devices into the single dipole magnetizer 112 includes slidably inserting it into the single dipole magnetizer 112 in the vertical orientation or the horizontal orientation.

In some embodiments, the method 200 further includes placing the medical device 140 or a second medical device of the plurality of medical devices in the horizontal orientation in the multipole magnetic field to impart a multipole magnetic signature (block 204). In some embodiments, placing the medical device 140 or the second medical device of the plurality of medical devices in the horizontal orientation in the multipole magnetic field includes lowering it into the trough 126. Other methods of placing the medical device 140 or the second medical device of the plurality of medical devices in the horizontal orientation in the multipole magnetic field are considered. In some embodiments, placing the medical device 140 or the second medical device of the plurality of medical devices in the horizontal orientation in the multipole magnetic field includes lowering it on the tray 128 into the trough 126.

The method 200 further includes removing the medical device 140 or the first and second medical devices of the plurality of medical devices from the magnetizer system 100 (block 206). In some embodiments, removing the medical device 140 or the first medical device of the plurality of medical devices from the magnetizer system 100 includes slidably removing it in the horizontal orientation or the vertical orientation from the cavity 114 of the single dipole magnetizer 112. In some embodiments, removing the medical device 140 or the second medical device of the plurality of medical devices from the magnetizer system 100 includes withdrawing it in the horizontal orientation from the multipole magnetic field. In some embodiments, withdrawing the medical device 140 or the second medical device of the plurality of medical devices in the horizontal orientation from the multipole magnetic field includes removing the tray 128 from the trough 126.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures can be made from the particular embodiments disclosed herein without departing from the scope of the concepts provided herein.

What is claimed is:

1. A method of imparting a magnetic signature, comprising:
inserting a first medical device into a single-dipole section of a magnetizer, wherein inserting the first medical device into the single-dipole section imparts a single-dipole magnetic signature with a first magnetic field;
positioning a second medical device into a multipole section of the magnetizer, wherein positioning the second medical device into the multipole section imparts a multipole magnetic signature with a second magnetic field.

2. The method according to claim 1, wherein the single-dipole section extends parallel to a longitudinal axis of the magnetizer, and wherein inserting the first medical device includes inserting a portion of the first medical device into a cavity of the magnetizer.

3. The method according to claim 1, wherein the multipole section of the magnetizer includes a trough extending parallel to a longitudinal axis of the magnetizer, and wherein positioning the second medical device includes positioning the second medical device in the trough.

4. The method according to claim 3, wherein positioning the second medical device in the trough comprises lowering a tray containing the second medical device into the trough along a transverse axis perpendicular to the longitudinal axis of the magnetizer.

5. The method according to claim 4, wherein lowering the tray comprises aligning one or more openings in the tray with one or more corresponding posts on the magnetizer.

6. The method according to claim 3, wherein a first plurality of magnets are embedded in the magnetizer body on a first side of the trough, wherein a second plurality of magnets are embedded in the magnetizer body on a second side of the trough opposite the first side of the trough, and wherein positioning the second medical device in the trough comprises positioning the second medical device between the first plurality of magnets and the second plurality of magnets.

7. The method according to claim 1, wherein the magnetizer comprises a radiofrequency identification (RFID) reader, the method further comprising reading an RFID tag associated with the first medical device and/or the second medical device by the RFID reader.

8. The method according to claim 1, wherein the magnetizer comprises one or more sensors, the method further comprising detecting the first medical device and/or the second medical device and communicating information related to the first medical device and/or the second medical device to an ultrasound probe.

9. The method according to claim 1, wherein the single-dipole section of the magnetizer is separated from the multipole section of the magnetizer, and wherein inserting the first medical device into the single dipole section of the magnetizer comprises aligning the first medical device with a transverse axis perpendicular to a longitudinal axis of the magnetizer.

10. The method according to claim 9, wherein the single-dipole section of the magnetizer comprises a cylindrical cavity, and wherein inserting the first medical device into the single dipole section of the magnetizer comprises inserting the first medical device into the cylindrical cavity.
